(19) United States
(12) Patent Application Publication
(10) Pub. No.: US 2002/0032387 A1
(43) Pub. Date: Mar. 14, 2002

(54) APPARATUS AND METHOD FOR REMOTE SPIROMETRY

(76) Inventors: JACOB GEVA, RISHON LEZION (IL); LEONID TRACHTENBERG, RISHON LEZION (IL); SERGEY YAKIREVITCH, BAT YAM (IL)

Correspondence Address: EITAN PEARL LATZER & COHEN-ZEDEK, ONE CRYSTAL PARK,SUITE 210, 2011 CRYSTAL DRIVE, ARLINGTON, VA, 22202-3709, US

(21) Appl. No.: 09/180,319

(22) PCT Filed: May 8, 1997
(86) PCT No.: PCT/IL97/00152

(30) Foreign Application Priority Data
May 8, 1996 (IL) 118191

(51) Int. Cl. A61B 5/091; A61B 5/08

(52) U.S. Cl. 600/538; 600/529; 128/204.23; 128/904

(57) ABSTRACT

An apparatus, which is illustrated in FIG. 6, includes a spirometer and oximeter for monitoring a patient's respiration, and the level of oxygen saturation in hemoglobin from a remote input unit (202) to an output unit (206) connected by a communication network.

### FIELD OF THE INVENTION

The present invention relates to spirometers and oxymeters generally and more particularly to apparatus and method for remote spirometry and oxymetry.

### BACKGROUND OF THE INVENTION

Spirometers as well as pulse oxymeters, for measuring oxygen saturation of arterial hemoglobin, are well known in the art and are used to determine parameters related to the functioning of lungs. As shown in FIG. 1 to which reference is now made, prior art spirometers, generally referenced 1, typically include a respiratory device 2 connected to an air flow sensor for sensing the respiration of patient 11, a converter 6 for converting the sensed respiration into a computer readable code and a computation unit 8, which typically includes a CPU 10 and a memory 12.

CPU 10 receives the converted sensed airflow of respiration and computes parameters related to the functioning of the lungs. The parameters typically include parameters obtained from Vital Capacity (VC) tests, Forced Vital Capacity (FVC) test and others, FEV₁, FEV₃, PEF, FEF 25%, FEF 50%, FEF 75% and others, all known in the art.

Prior art spirometers typically also include a keyboard 14, a display 16 and a printer 20 to enable data and command input and to display or print the results of the tests, respectively.

Prior art oxymeters come in various configurations for various purposes such as finger probe, nasal bridge sensor, pediatric adhesive sensor or adult adhesive sensor, all of which are widely used.

A conventional prior art pulse oxymeter finger probe has two LEDs. One led transmits infra-red light at a wave length of approximately 940 nm and the other transmits red light at a wave length of approximately 660 nm. The absorption of these wave lengths of light in living tissues significantly different for oxygenated hemoglobin (HbO₂) and reduced hemoglobin. From this known relationship, the HbO₂ level can be calculated. A photo sensor measures the absorption of these wave lengths of light passing through the tested tissue. The percentage of saturation of oxygen on hemoglobin can be calculated from the ratio of red light and infra red light which are detected at the photo sensor.

Prior art pulse oxymeters may be stationary or portable. In both cases, the system is operated by a physician or a paramedic trained to operate them. Therefore, the patient and the operator have to meet, in the hospital or in the physician's clinic or in the patient's residence in order to perform the test.

Breathing into a spirometer can be performed in a variety of ways. The manner of breathing has to be taken into consideration together with the data produced by the spirometer. Normally, when a physician and a patient meet and conduct a test, the physician is able to monitor the patient's fashion of breathing, take it into consideration and correct it if necessary.

In the art there are also known spirometry systems which employ telephone communication between the patient and the physician as disclosed in U.S. Pat. No. 4,296,756 to Dauning et al. According to the system disclosed by Dauning, the patient breaths into a transducer, which measures air flow and pressure. These measurements are converted to digital data. The data is transmitted via a telephone line to a remote central computer, where the physician is located. In such a system the physician has no knowledge regarding the way in which the patient is breathing; the physician receives only the data produced by the spirometer.

A similar system is a monitoring system disclosed in PCT application no. PCT/AU89/00261 to Morgan J. Mark et al. This system is a portable pocket size device comprising a spirometer and a data accumulation unit which is adapted to connect to a computer, the computer including a modem. First, the patient breathes into the device. The device's spirometer measures the breath and this data is stored in the accumulation unit. Afterwards, the device is connected to a computer via a computer interface for transmitting the data to a remote location via a telephone communication network by using the computer's modem.

### SUMMARY OF THE INVENTION

It is an object of the present invention to provide apparatus for remote pulse oxymetry and spirometry.

It is also an object of the present invention to provide an improved method for spirometry.

It is a further object of the present invention is to provide a method and apparatus for spirometry, in which a patients way of breathing can be monitored from a remote location.

According to a preferred embodiment of the present invention there is thus provided a two part apparatus for remote spirometry, where one part is located at the patient end and the other is located at the operator's end. The operator is typically a physician or a paramedic. The two parts are being connected via any communication network,- preferably the telephone network.

In accordance with the present invention, the apparatus for remote spirometry includes an input unit upstream of the communication network, and an output unit downstream the communication network.

According to a preferred embodiment, there is provided an apparatus for remote spirometry and oxymetry including a remote input unit, located at one end of a communication network, for receiving at least one respiration and for producing a transmittable signal therefrom and an output unit, located at a second end of the communication network, for receiving the signal and for generating a pulmonary test result and an oxymetry test result therefrom.

The remote input unit includes at least one mechanical sensor and at least one oxygen saturation sensor. The mechanical sensor for measuring predetermined mechanical properties of the respiration and the oxygen saturation sensor for producing at least one measurement of the level of oxygen saturation in hemoglobin.

With respect to another preferred embodiment of the invention of the invention there is thus provided an apparatus for remote spirometry constructed in accordance with another preferred embodiment of the invention comprising: a remote input unit, located at one end of a communications network, for receiving at least one respiration from a user and for producing a transmittable signal therefrom and an output unit, located at a second, end of the communication network, for receiving the signal, for generating a pulmonary test result therefrom.

The remote input unit comprises: an input device into which the at least one respiration is respired, a sensing unit for performing at least one type of measurement of the respiration, a video camera for detecting the images of the user producing the at least one respiration and a communications interface for connecting to the communications network.

The output unit includes: a communications interface unit for connecting to the communications network, a processing unit for processing signals received from the remote input unit into a pulmonary test result and a display for displaying the pulmonary test result and images of the user producing the respiration.

In addition, the sensing unit are adapted for converting the measurement into a first electrical signal and the video camera converting the images into a second electrical signal.

According to yet a further aspect of the invention the input unit further includes an analog to digital converter for providing a digital signal from the first and the second electrical signals and a processor for producing the transmittable signal from the digital signal.

According to another aspect of the invention the output unit further includes a video camera for detecting images of a second user producing the respiration and the remote input unit further comprises a display for displaying the images of the second user.

According to another aspect of the invention the mechanical sensor is adapted for converting the respiration into a first electrical signal, the oxygen saturation sensor is adapted for converting the measurement of the level of oxygen saturation in hemoglobin, into a second electrical signal, the input unit further comprises, an input device into which the respiration is respired, an analog to digital converter for providing a first digital signal from the first and the second electrical signals, a pulse oxymeter unit for providing a second digital signal from the second signal and a CPU for producing the transmittable signal from the first and the second digital signals.

Additionally an apparatus according to yet another embodiment of the invention can also comprise an amplifier for amplifying the first and the second electrical signals.

Furthermore, an apparatus according to the invention may also include a storage unit for storing the transmittable signal, thereby enabling the CPU to transmit the transmittable signal independently from the production thereof.

Accordingly, the communication network can be a telephone network.

With respect to yet a different aspect of the invention the input unit also comprises a speaker for generating an audio signal from the transmittable signal for transmitting it via the telephone network.

Furthermore, the input unit includes a modem for transmitting the transmittable signal via the telephone network.

Additionally, the transmittable signal may include an unprocessed digital signal.

Alternatively, the transmittable signal may include a processed signal.

Accordingly the transmittable signal may include a partially processed, partially unprocessed signal.

According to yet another embodiment of the present invention, the output unit includes a CPU for generating the pulmonary test result and the oxymetry test result and means for displaying the pulmonary test result and the oxymetry test result.

Alternatively, the output unit includes a frequency demodulator for converting the transmitted audio signal into a digital signal, a CPU for receiving the digital signal, for generating the pulmonary test result therefrom and the oxymetry test result and a display unit for displaying both the pulmonary test result and the oxymetry test result.

According to yet a further embodiment of the invention, the output unit includes a modem for receiving the transmittable signal a CPU for generating the pulmonary test result and the oxymetry test result from the signal received by the modem and a display for displaying the pulmonary test result and the oxymetry test result.

According to yet a different embodiment of the invention there is provided a method for remote spirometry and oxymetry comprising: producing, at a remote location, a transmittable signal from at least one respiration, the transmittable signal relating to at least one mechanical property and to the a level of oxygen saturation in hemoglobin test, transmitting the transmittable signal via a communication network to another location and receiving, downstream of the communication network, the transmittable signal and generating a pulmonary test result and an oxymetry test result therefrom.

In addition, according to another embodiment of the invention the step of producing includes:

respiring at least one respiration.

measuring at least one mechanical property of the respiration.

converting the at least one mechanical property into a first electrical signal.

measuring the level of oxygen saturation in hemoglobin during the respiration.

converting the level of oxygen saturation in hemoglobin into a second electrical signal.

converting the first electrical signal and the second electrical signal into a digital signal.

producing the transmittable signal from the digital signal.

In addition, the method according to an alternate embodiment of the invention, includes the step of amplifying the electrical signals before the step of converting.

With respect to another aspect of the present invention, the method according to the invention includes the step of storing the transmittable signal after the step of producing, thereby enabling to transmit the transmittable signal independently from the production thereof.

Additionally, in a method according to yet a further embodiment of the invention, the communication network is a telephone network.

Furthermore, the method includes the step of generating an audio signal from the transmittable signal and transmitting the audio signal via the telephone network.

In addition, the method may also include the step of transmitting the transmittable signal through a modem via the telephone network.

With respect to another aspect of the invention, the method includes the step of either forming the transmittable signal as an unprocessed signal, forming the transmittable signal as a processed signal or forming the transmittable signal as a partially processed partially unprocessed signal.

Alternatively, the method according to the invention includes the steps of generating comprising processing the received signal, displaying and printing the results.

According to yet a further embodiment of the invention the method of the invention includes the step of demodulating downstream the telephone network the audio signal so as to enable receiving the transmitted signal.

Additionally, the audio signal is transmitted via a telephone microphone to the telephone network.

In addition the apparatus also includes an amplifier for amplifying the first and the second electrical signals.

### BRIEF DESCRIPTION OF THE DRAWINGS

The present invention will be understood and appreciated more fully from the following detailed description taken in conjunction with the appended drawings in which:

FIG. 1 is a partially pictorial, partially block diagram illustration of a prior art spirometer;

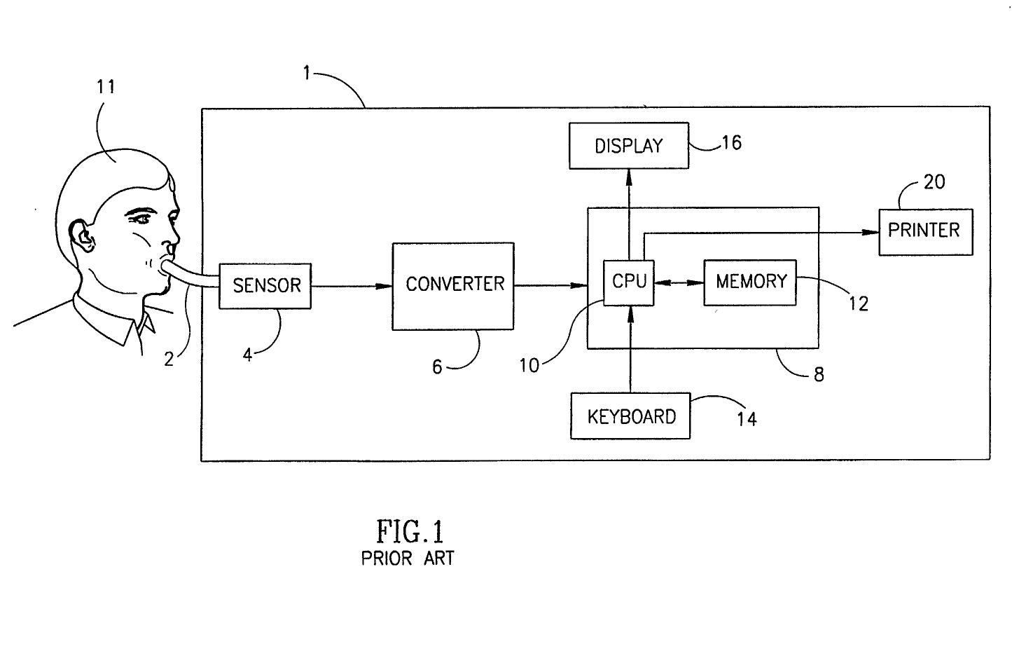

FIG. 2 is a partially pictorial, partially block diagram illustration of a apparatus for remote spirometry, constructed and operative in accordance with a preferred embodiment of the present invention;

FIG. 3 is a partially pictorial, partially block diagram illustration of a apparatus for remote spirometry, constructed and operative in accordance with a second preferred embodiment of the present invention;

FIG. 4 is a partially pictorial, partially block diagram illustration of a apparatus for remote spirometry, constructed and operative in accordance with a third preferred embodiment of the present invention;

FIG. 5 is a partially pictorial, partially block diagram illustration of a apparatus for remote spirometry, constructed and operative in accordance with a fourth preferred embodiment of the present invention; and

FIG. 6 is a partially pictorial, partially block diagram illustration of the apparatus of FIG. 5.

### DETAILED DESCRIPTION OF THE PRESENT INVENTION

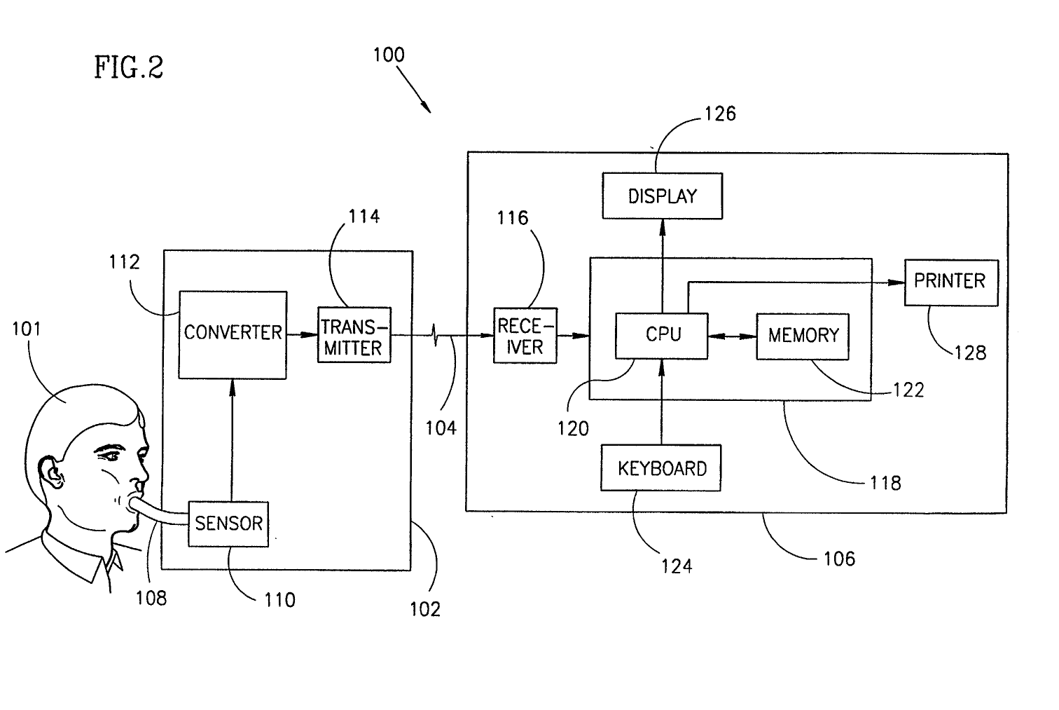

Reference is now made to FIG. 2 which is a partially pictorial, partially block diagram illustration of an apparatus for advanced remote spirometry, constructed and operative in accordance with a preferred embodiment of the present invention.

The apparatus for advanced remote spirometry, generally referenced 100, is a two part system comprising an input unit 102, located upstream of a communication network, schematically indicated by the line referenced 104, and an output unit 106, located downstream of the communication network 104.

It is a particular feature of the present invention that the apparatus for advanced remote spirometry 100 is divided into two units which are remote from each other. Therefore, the input unit can be located at the patient home whereas the output unit can be located in his physician's clinic.

The input unit 102 preferably comprises an air input device 108 into which a patient 101 respires, a sensing unit 110 which includes an air flow sensor. The air flow sensors preferably, but not necessarily, is an air pressure sensor. Input unit 102 also includes a converter 112 which converts the signal provided by the sensing unit 110 into a transmittable signal which is in turn, transmitted by a transmitter 114 via the communication network 104 to the output unit 106.

The output unit 106 preferably comprises a receiver 116 which receives the transmitted signal and directs it to a computation unit 118. The computation unit 118 preferably comprises a CPU 120 which processes the signal received via the communication network and outputs the results of the pulmonary test conducted by the patient 101 in a remote location therefrom.

The output unit 106 preferably also comprises a keyboard 124 to enable the output unit operator, typically a physician or a paramedic, to input commands and data to the CPU 120, a display 126 and a printer 128 for displaying and printing the pulmonary and oxymetry test results, respectively.

The computation unit 118 and the display 126 may be any suitable computation units, for example part of a personal computer, such as an International Business Machine (IBM) personal computer, which includes a CPU, such as an Intel Inc. 40486 CPU. The printer 128 may be any commercial printer connected thereto, such as the printers commercially available from Hewlett Packard of California, U.S.A.

It is a particular feature of the present invention that the signal sent from the transmitter 114 to the receiver 116 via the communication network includes either the digital representation of the raw data gathered by the sensing unit 110 or data which has been processed to a desired extent by the input unit 106 as described in detail hereinbelow.

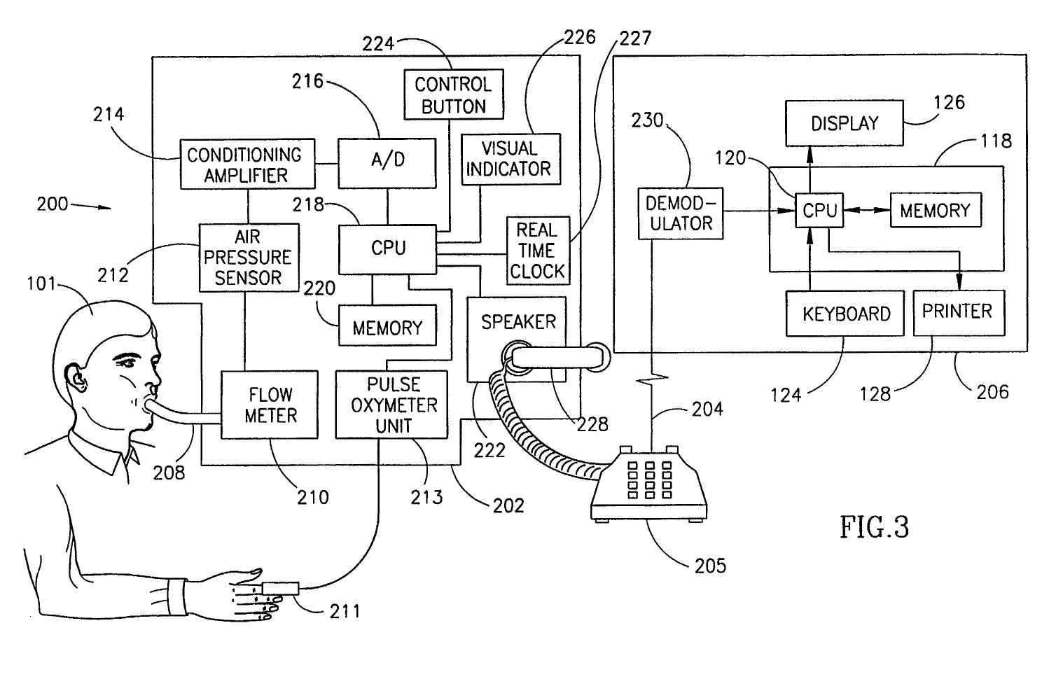

According to one preferred embodiment of the present invention, as shown in FIG. 3 to which reference is now made, the communication network 104 is the telephone network, schematically indicated by the telephone network line 204 and the telephone set 205.

The apparatus for remote spirometry and oxymetry of FIG. 3, generally referenced 200, preferably includes an input unit 202 which is connected to the output unit 206 via the telephone 205 connected to the telephone network 204. As with the apparatus for remote spirometry 100, the input unit 202 and the output unit 206 are remote one from each other.

The input unit 202 preferably comprises an air input device 208, an air pressure sensor 212, an oxygen saturation sensor 211 and pulse oxymetry unit 213. The air input device 208 includes preferably a tube, which preferably includes a flow meter 210 which operates to create a pressure difference, in a manner well known in the art, so as to provide an input to air pressure sensor 212.

In the present example oxygen saturation sensor 211 is a finger probe type sensor, manufactured and sold by a division of Palco Inc. of California, U.S.A.

The air pressure sensor 212 compares the pressure induced by the respired air to the ambient air pressure and outputs an electrical current output, of a typical frequency between 1-5 Hz to a conditioning amplifier 214. A suitable air pressure sensor is the 160 PC low pressure sensor, manufactured and sold by Micro Switch, a division of Honeywell Inc. of California, U.S.A.

The conditioning amplifier 214 amplifies the output signals of the air pressure sensor 212 to a level accepted by an analog to digital converter (A/D) 216. Via A/D converter 216, the conditioning amplifier 214 outputs a digital signal, typically at a sampling rate of 400 per second, to a central processing unit (CPU) 218 which may be any suitable CPU, such as the CMOS single-chip 8-bit Intel-80C31 micro-controller, manufactured and sold by Intel Co. of California, USA.

According to one preferred embodiment of the present invention the CPU 218 processes the signal received from the A/D 216 and stores in a memory 220 results of the pulmonary and oxymetry tests. Alternatively, the CPU directs the received signal as such to the memory 220, or processes the signal and directs both the raw signal and the processed results to the memory 220.

Oxygen saturation sensor 211 is connected to pulse oxymeter unit 213. Pulse oxymeter unit 213 controls oxygen saturation sensor 211 and processes the signals provided therefrom into data relating to the oxygen level. This data is transferred to CPU 218 which directs it to memory 220.

The input unit 202 preferably also comprises one or more control buttons 224 which enables the patient who performs the test to control to a desired extent the operation of the input unit 202, as described in detail hereinbelow. The input unit 202 also comprises one or more visual indicators 226, such as light emitting diodes (LEDs), alpha numeric or graphic displays, which indicate to the patient 101 in which state the input unit is. The control buttons 224 and the visual indicators 226 are connected to the CPU 218.

According to an alternative embodiment of the present invention the CPU 218 receives the signal from the AND 216 or from pulse oxymeter unit 213 and directs it before or after processing to a speaker 222. The speaker 222 may be any suitable speaker which receives a digital signal generated by the CPU 218 and transmits therefrom an audio signal. An example of a suitable speaker is the AT-13, manufactured and sold by Project Unlimited Inc. of Ohio, USA. The carrier frequency of the transmitted signal generated by the CPU 218 via the speaker 222 is typically 1700 Hz and the frequency modulation thereof is ±500 Hz.

The input system 202 preferably also comprises a real time clock 227 connected to the CPU 218 for indicating the actual time at which the test was performed and the time at which the data is transmitted to the output unit 206. The system preferably also comprises a temperature sensor (not shown) for providing the ambient temperature when a temperature correction of is desired.

It is a particular feature of the present invention that, unlike prior art apparatus for oxymetry and remote spirometry, the input unit 202 may be used to at least partially process the signal resulting from either the pulmonary and oxymetry test.

According to the invention, air pressure sensor 212 may include a variety of mechanical and physical sensors as well as other types of oxymated hemoglobin for obtaining a lot of information from the respired air, indicating as to the physical condition of the patient.

According to one preferred embodiment of the present invention the patient 101 operates the input unit 202 as follows:

A. The patient 101 presses the control button 224 to indicate to CPU 218 to reset the input unit 202 for a new test. Additionally, the CPU 218 may initiate a self test of the input unit 202 to verify its readiness.

B. After the CPU 218 resets the input unit 202, it directs a ready signal to the LEDs 226 which indicate the same to the patient.

C. The patient connects to oxygen saturation sensor 211 and blows into the air input device 208.

D1. The air blow is converted as described hereinabove via the flow meter 210, the air pressure sensor 212 and amplifier 214 and the A/D 216 to a digital signal provided to the CPU 218.

D2. CPU 218 receives the signal from pulse oxymeter unit 213 and stores it in memory 220.

E1. According to one preferred embodiment of the present invention the CPU 218 calculates the parameters indicative to the test such as FVC and VC and stores the results in the memory 220.

E2. Alternatively to step E1 or in a desired combination therewith, the CPU 218 stores the raw signal in the memory 220.

F. The LEDs 226 indicate that the data of steps E1 or E2 or a combination thereof are stored in the memory 220.

G. The patient dials a number using his telephone 205 so as to connect to the output unit 206 which is remote therefrom, such as in his physician's clinic.

H. The patient puts the telephone's 205 handset microphone 228 on the input unit speaker 222 and presses the control buttons 224 so that the CPU 218 retrieves the stored signal from the memory 220 and transmits it via speaker 222 to the telephone handset microphone 228 and therefrom via the network 204 to the output unit 206.

It will be appreciated that the test itself (steps A-F) and the transmission of the data or the calculated results thereof (steps G-H) need not occur at the same time. For example, if the patient does not feel well or if the respiration required for the test tires him, he may transmit the results (steps G-H) later.

According to a preferred embodiment of the present invention, the test itself is a multi-blow test. According to this embodiment, step A-H described hereinabove are preceded by a self test of the input unit. According to this alternative embodiment, steps C and are repeated, for example twice, so as to provide multiple sets of test data signals to the CPU 218.

It will be appreciated that the data of the pulmonary and oxymetry tests may be also sent directly, without storing it in the memory 220, via the speaker 222 as described hereinabove.

The output unit 206 preferably comprises a frequency demodulator 230 which receives the modulated audio frequency signal sent from the input unit 202 via the telephone network 204 and produces therefrom a computer readable code. The computer readable code is directed to the output unit CPU, which may be similar to the CPU 120 (FIG. 2).

It will be appreciated that the CPU 120 is part of a computation unit which, like the display and the printer of the output unit 206, may be similar to the computation unit 118 and therefore, similar reference numerals are used in for the computation unit 118, the display 126 and the printer 128 in FIGS. 2 and 3.

Using the output unit 206 the physician or the paramedic receiving the test results via telephone may analyze any display or print any parameter or graph related to the pulmonary and oxymetry tests conducted by the patient in a remote location, and if required, may instruct the patient over the telephone whether any measures, such as consuming a medicine, should by taken.

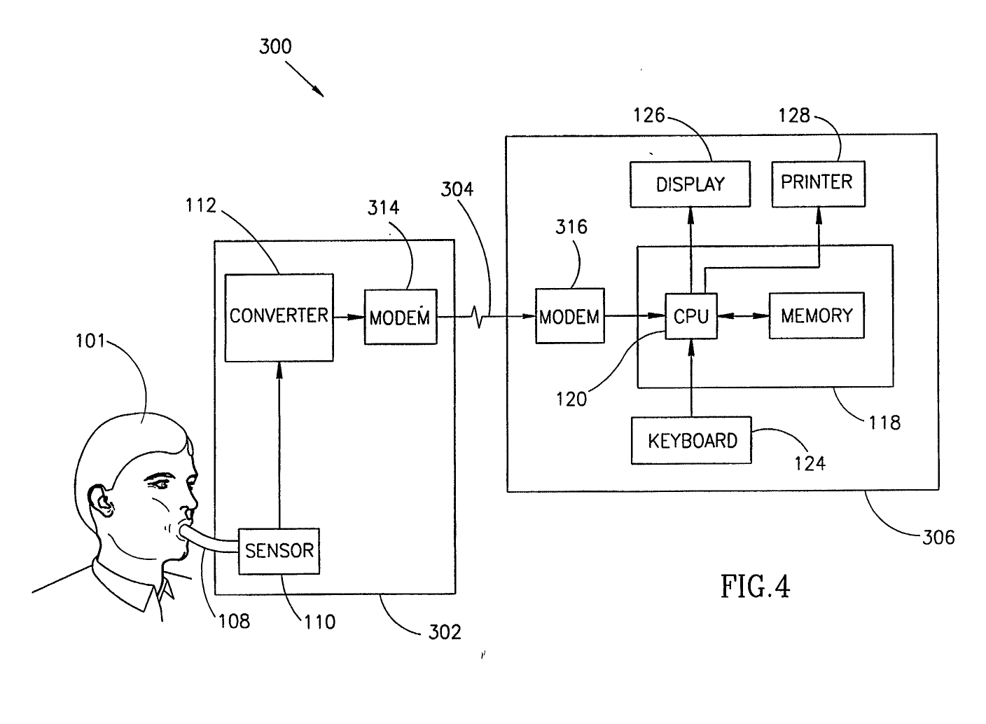

Reference is now made to FIG. 4 which is a partially pictorial, partially block diagram illustration of an advanced remote apparatus for spirometry, constructed and operative in accordance with a third preferred embodiment of the present invention.

The apparatus for remote spirometry, generally referenced 300, illustrates transmission and receipt of the pulmonary test data signal via modems.

The apparatus 300 preferably comprises an input unit 302 and an output unit 306 connected via the telephone network 304.

In the illustrated embodiment, the apparatus 300 comprises all the elements of the apparatus 100 but the receiver and the transmitter are modem 314 and modem 316, respectively, and the communication network 104 is the telephone network 304.

It will be appreciated that the input unit 302 may also include all the elements of the input unit 202, except for the speaker 222. According to this preferred embodiment, the CPU 218 directs the transmitted data directly to the modem 314 which sends it via the telephone network 304 to the modem 316 for processing in the output unit 306.

It will be appreciated that the preferred embodiments described hereinabove are described by way of example only and that numerous modifications thereto, all of which fall within the scope of the present invention, exist. For example, the telephone network 204 may be a radio-telephone network, a direct link telephone system or a cellular telephone network and accordingly, the telephone 205 may be a cellular phone.

Another example is the use of an analog demodulator instead of the digital demodulator 230 coupled with an AID converter for converting the received signal to a computer readable code.

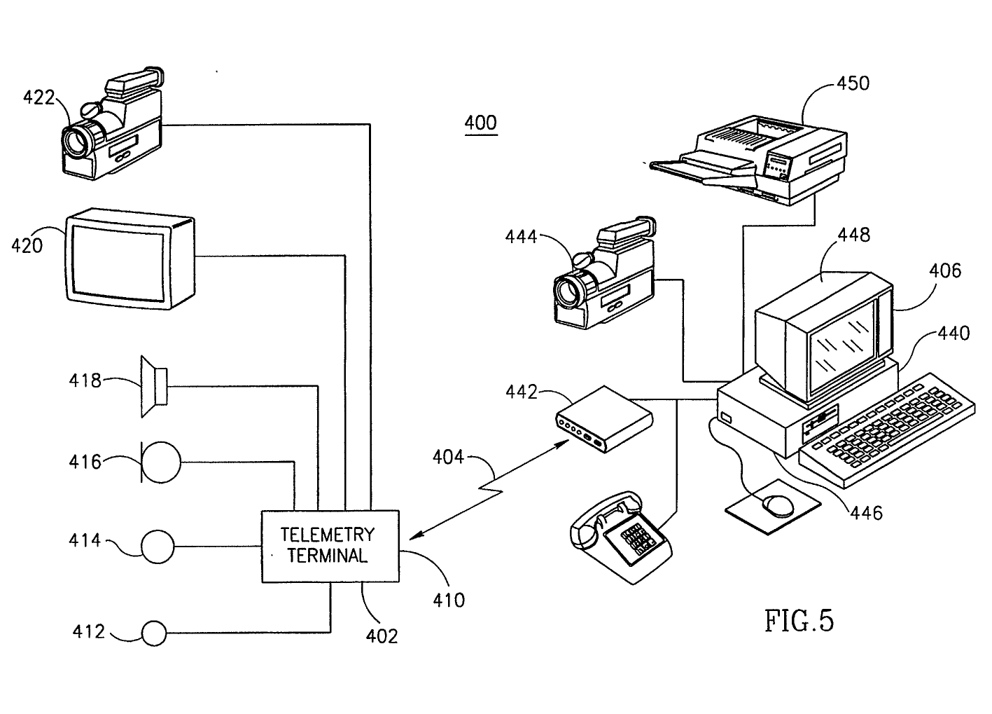

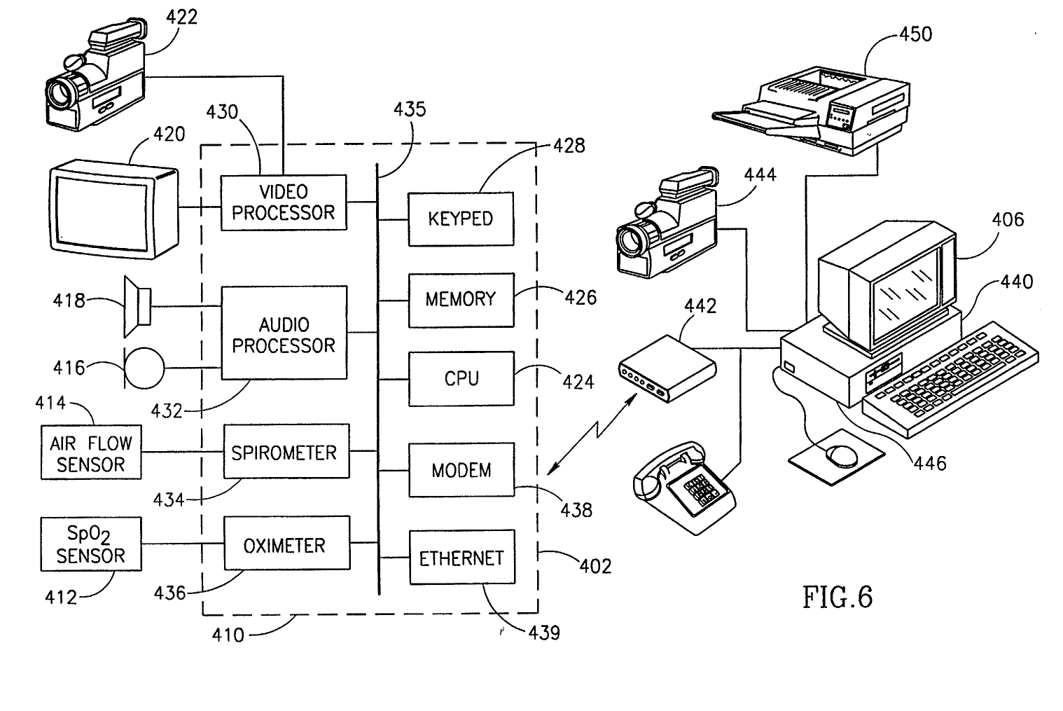

According to another aspect of the invention, the apparatus furthermore includes video capabilities, providing the physician with a view of the patient, using the system. Reference is now made to FIGS. 5 and 6 which are partially pictorial, partially block diagram illustrations of an apparatus, generally referenced 400, for remote spirometry, constructed and operative in accordance with a fourth preferred embodiment of the present invention.

The apparatus 400 preferably comprises an input unit 402 and an output unit 406, connected via a communications network 404.

In the illustrated embodiment, the input unit 402 comprises a telemetry terminal 410 which is connected to an oxymeter sensor 412, a spirometer air flow sensor 414, a microphone 416, a speaker 418, a video monitor 420 and a video camera 422.

As shown in FIG. 6, the telemetry terminal 410 includes a Central Processing Unit (CPU) 424, memory 426, a key pad 428, a video processor 430, an audio processor 432, a spirometer interface 434, an oxymeter interface 436, a modem 438 for connecting to a telephone network and an Ethernet interface 439 for connecting to a digital communications network. All of the elements of telemetry terminal 410 are interconnected via a data bus 435.

Video processor 430 processes the video signal provided by video camera 422 into a signal which can be transmitted to output unit 406. Output unit 406 receives and processes and image signal from the signal received from input unit 402 and provides the processed image signal to video screen 420.

Audio processor 432 processes and audio signal, provided by microphone 416, into a signal which can be transmitted to output unit 406. Audio processor 432 also provides an audio signal to speaker 418 from a signal which is received from output unit 406.

The output unit 406 comprises a computing unit 440 connected to transmitting and receiving unit, which in the present example is a modem 442, a video camera 444, and a printer 450. Computing unit 440 includes a computer 446 and a monitor 448.

The apparatus 400 provides a two way video link between the input unit 402 and the output unit 406. Accordingly a patient and a physician can see one another. Another aspect of this apparatus 400 is remote oxygen level monitoring.

As the patient performs a breathing test, his air flow is measured by spirometer air flow sensor 414, and his oxygen level is measured by the oxymeter sensor 412. At the same time, images of the patient breathing, are detected by the video camera 422. All of this information is converted to digital data at the telemetry terminal and transmitted via the communications network 404.

Received at the output unit 406, the information relating to the spirometer measurements and the oxymeter measurements is processed and produced for display, either on screen by monitor 448 or printer 450. The information relating to the images of the patient, which were taken by video camera 422 are displayed on monitor 448. A physician using the output unit 406 can thus monitor the patients manner of breathing and take it into consideration.

In the opposite direction, the physician can use the video camera 444 to demonstrate the right manner of breathing. As he sets an example of a proper manner of breathing, images of the patient breathing, are detected by the video camera 444. This video signal is provided to video monitor 420 for display. This way, the patient can see the physician, learn and correct his manner of breathing.

It will be appreciated by persons skilled in the art that the present invention is not limited to what has been particularly shown and described hereinabove. Rather, the scope of the present invention is defined only by the claims that follow:

1. Apparatus for remote spirometry and oxymetry comprising:
a remote input unit, located at one end of a communication network, for receiving at least one respiration and for producing a transmittable signal therefrom; and
an output unit, located at a second end of said communication network, for receiving said signal and for generating a pulmonary test result and an oxymetry test result therefrom,
wherein said remote input unit includes at least one mechanical sensor and at least one oxygen saturation sensor, said mechanical sensor for measuring predetermined mechanical properties of said at least one respiration and said oxygen saturation sensor for producing at least one measurement of the level of oxygen saturation in hemoglobin.

2. Apparatus according to claim 1 wherein:
said mechanical sensor is adapted for converting said at least one respiration into a first electrical signal;
said oxygen saturation sensor is adapted for converting said at least one measurement of the level of oxygen saturation in hemoglobin into a second electrical signal;
said input unit further comprises:
an input device into which said at least one respiration is respired;
an analog to digital converter for providing a first digital signal from said first and said second electrical signals;
a pulse oxymeter unit for providing a second digital signal from said second signal; and
a CPU for processing said transmittable signal from said first and said second digital signals.

3. Apparatus according to claim 2 and also comprising an amplifier for amplifying said first and said second electrical signals.

4. Apparatus according to claim 2 and also comprising:
a storage unit for storing said transmittable signal, thereby enabling said CPU to transmit said transmittable signal independently from the production thereof.

5. Apparatus according to claim 1 wherein said communication network is a telephone network.

6. Apparatus according to claim 5 wherein said input unit also comprises a speaker for generating an audio signal from said transmittable signal for transmitting it via said telephone network.

7. Apparatus according to claim 5 wherein said input unit comprises a modem for transmitting said transmittable signal via said telephone network.

8. Apparatus according to claim 1 wherein said transmittable signal is an unprocessed digital signal.

9. Apparatus according to claim 1 wherein said transmittable signal is a processed signal.

10. Apparatus according to claim 1 wherein said transmittable signal is partially processed, partially unprocessed signal.

11. Apparatus according to claim 1 wherein said output unit comprises:
a CPU for generating said pulmonary test result and said oxymetry test result; and
means for displaying said pulmonary test result and said oxymetry test result.

12. Apparatus according to claim 6 and wherein said output unit comprises:
a frequency demodulator for converting the transmitted audio signal into a digital signal;
a CPU for receiving said digital signal and for generating said pulmonary test result therefrom and said oxymetry test result; and
means for displaying said pulmonary test result and said oxymetry test result.

13. Apparatus according to claim 7 and wherein said output unit comprises:
a modem for receiving said transmittable signal;
a CPU for generating said pulmonary test result and said oxymetry test result from the signal received by said modem; and
means for displaying said pulmonary test result and said oxymetry test result.

14. A method for remote spirometry and oxymetry comprising:
producing, at a remote location, a transmittable signal from at least one respiration, said transmittable signal relating to at least one mechanical property and to the a level of oxygen saturation in hemoglobin test;
transmitting said transmittable signal via a communication network to another location; and
receiving, downstream of said communication network, said transmittable signal and generating a pulmonary test result and an oxymetry test result therefrom.

15. A method according to claim 14 wherein said step of producing comprising:
respiring at least one respiration;
measuring at least one mechanical property of said at least one respiration;
converting said at least one mechanical property into a first electrical signal;
measuring the level of oxygen saturation in hemoglobin during said at least one respiration;
converting said level of oxygen saturation in hemoglobin into a second electrical signal;
converting said first electrical signal and said second electrical signal into a digital signal; and
producing said transmittable signal from said digital signal.

16. A method according to claim 15 comprising, amplifying said electrical signals before said step of converting.

17. A method according to claim 14 comprising, storing said transmittable signal after said step of producing, thereby enabling to transmit said transmittable signal independently from the production thereof.

18. A method according to claim 14 wherein said communication network is a telephone network.

19. A method according to claim 18 comprising, generating an audio signal from said transmittable signal and transmitting said audio signal via said telephone network.

20. A method according to claim 18 comprising, transmitting said transmittable signal through a modem via said telephone network.

21. A method according to claim 14 comprising, forming said transmittable signal as an unprocessed signal.

22. A method according to claim 14 comprising, forming said transmittable signal as a processed signal.

23. A method according to claim 14 comprising, forming said transmittable signal as a partially processed partially unprocessed signal.

24. A method according to claim 14 wherein said step of generating comprising processing said received signal.

25. A method according to claim 14 comprising, displaying said result.

26. A method according to claim 14 comprising, printing said results.

27. A method to claim 18 comprising, demodulating downstream said telephone network said audio signal so as to enable receiving said transmitted signal.

28. A method according to claim 19 wherein said audio signal is transmitted via a telephone microphone to said telephone network.

29. Apparatus for remote spirometry comprising:
a remote input unit, located at one end of a communications network, for receiving at least one respiration from a user and for producing a transmittable signal therefrom; and
an output unit, located at a second, end of said communication network, for receiving said signal, for generating a pulmonary test result therefrom,
wherein said remote input unit comprises:
an input device into which said at least one respiration is respired;
sensing means for performing at least one type of measurement of said at least one respiration;
a video camera for detecting the images of said user producing said at least one respiration, and
communications interface means for connecting to said communications network;
said output unit comprises:
communications interface means for connecting to said communications network;
processing means for processing signals received from said remote input unit into a pulmonary test result;
display means for displaying said pulmonary test result and images of said user producing said at least one respiration

30. Apparatus according to claim 29 wherein:
said sensing means are adapted for converting said at least one measurement into a first electrical signal;
said video camera converting said images into a second electrical signal.

31. Apparatus according to claim 29 wherein said input unit further comprises:
an analog to digital converter for providing a digital signal from said first and said second electrical signals; and
a processor for producing said transmittable signal from said digital signal.

32. Apparatus according to claim 29 wherein:
said output unit further comprises a video camera for detecting images of a second user producing at least one respiration, and
said remote input unit further comprises display means for displaying said images of said second user.

33. Apparatus according to claim 30 and also comprising an amplifier for amplifying said first and said second electrical signals.

34. Apparatus according to claim 30 and also comprising:
a storage unit for storing said transmittable signal, thereby enabling said processor to transmit said transmittable signal independently from the production thereof.

35. Apparatus according to claim 29 wherein said communication network is a telephone network.

36. Apparatus according to claim 34 wherein said input unit also comprises a speaker for generating an audio signal from said transmittable signal for transmitting it via said telephone network.

37. Apparatus according to claim 34 wherein said input unit comprises a modem for transmitting said transmittable signal via said telephone network.

38. Apparatus according to claim 29 wherein said transmittable signal is an unprocessed digital signal.

39. Apparatus according to claim 29 wherein said transmittable signal is a processed signal.

40. Apparatus according to claim 29 wherein said transmittable signal is partially processed, partially unprocessed signal.

41. Apparatus according to claim 29 wherein said output unit comprises:
a processor for generating said pulmonary test result; and
means for displaying said pulmonary test result.

42. Apparatus according to claim 36 and wherein said output unit comprises:
a frequency demodulator for converting the transmitted audio signal into a digital signal;
a processor for receiving said digital signal and for generating said pulmonary test result therefrom; and
means for displaying said pulmonary test result.

43. Apparatus according to claim 36 and wherein said output unit comprises:
a modem for receiving said transmittable signal;
a processor for generating said pulmonary test result from the signal received by said modem; and
means for displaying said pulmonary test result.

44. A method for remote spirometry comprising:
detecting, at a remote location, images of a user producing at least one respiration;
producing, at said remote location, a transmittable signal from said at least one respiration, said transmittable signal relating to at least one property of said at least one respiration and to said detected images;
transmitting said transmittable signal via a communication network to another location; and
receiving, downstream of said communication network, said transmittable signal and generating a pulmonary test result and video images therefrom.

45. A method according to claim 44 wherein said step of producing comprising:
respiring at least one respiration;
measuring at least one property of said at least one respiration;
converting said at least one property into a first electrical signal;
converting said detected images into a second electrical signal;
converting said first electrical signal and said second electrical signal into a digital signal; and
producing said transmittable signal from said digital signal.

46. A method according to claim 45 comprising, amplifying said electrical signals before said step of converting.

47. A method according to claim 44 comprising, storing said transmittable signal after said step of producing, thereby enabling to transmit said transmittable signal independently from the production thereof.

48. A method according to claim 44 wherein said communication network is a telephone network.

49. A method according to claim 48 comprising, generating an audio signal from said transmittable signal and transmitting said audio signal via said telephone network.

50. A method according to claim 48 comprising, transmitting said transmittable signal through a modem via said telephone network.

51. A method according to claim 44 comprising, forming said transmittable signal as an unprocessed signal.

52. A method according to claim 44 comprising, forming said transmittable signal as a processed signal.

53. A method according to claim 44 comprising, forming said transmittable signal as a partially processed partially unprocessed signal.

54. A method according to claim 44 wherein said step of generating comprising processing said received signal.

55. A method according to claim 44 comprising, displaying said result.

56. A method according to claim 44 comprising, printing said results.

57. A method to claim 48 comprising, demodulating downstream said telephone network said audio signal so as to enable receiving said transmitted signal.

58. A method according to claim 49 wherein said audio signal is transmitted via a telephone microphone to said telephone network.

59. An apparatus according to any of claims 1-13 and 29-43, substantially as illustrated in any of the drawings.

60. An apparatus according to any of claims 1-13 and 29-43, substantially as described hereinabove.

61. A method according to any of claims 14-28 and 44-58, substantially as illustrated in any of the drawings.

62. A method according to any of claims 14-28 and 44-58, substantially as described hereinabove.
